(19) United States
(12) Patent Application Publication
(10) Pub. No.: US 2026/0216498 A1
(43) Pub. Date: Jul. 30, 2026

(54) BLOOD PUMP WITH RESORBABLE ATRAUMATIC TIP

(71) Applicant: ABIOMED, Inc., Danvers, MA (US)

(72) Inventors: Christopher Sheils, Danvers, MA (US); Daniel Raess, Danvers, MA (US)

(73) Assignee: ABIOMED, Inc., Danvers, MA (US)

(21) Appl. No.: 19/460,603

(22) Filed: Jan. 27, 2026

Related U.S. Application Data
(60) Provisional application No. 63/749,892, filed on Jan. 27, 2025.

(51) Int. Cl. A61M 60/90; A61M 60/122; A61M 60/80

(52) U.S. Cl. CPC A61M 60/90; A61M 60/122; A61M 60/80

(57) ABSTRACT

Resorbable and/or dissolvable atraumatic tips and methods of using atraumatic tips are provided. The atraumatic tip includes a proximal portion and a distal portion. The proximal and/or distal portion may be made of a material configured to be resorbable and/or dissolvable in the blood of a patient. The atraumatic tip may be attachable to a blood pump assembly.

### CROSS-REFERENCE TO RELATED APPLICATIONS

The present application claims priority to U.S. Provisional Patent Application 63/749,892, filed Jan. 27, 2025, the contents of which are incorporated be reference herein in its entirety.

### TECHNICAL FIELD

Embodiments disclosed herein relate to medical devices, such as intravascular blood pumps, devices and methods.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the Impella® heart pump. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### SUMMARY OF INVENTION

In some embodiments, an atraumatic tip for a blood pump system may be provided. The atraumatic tip may include a proximal portion attachable to a blood pump and a distal portion. At least the distal portion of the atraumatic tip may be configured to be resorbable and/or dissolvable into a body of a patient.

In some embodiments, a blood pump system may be provided. The blood pump system may include a blood pump. The blood pump may include a cannula, a blood flow inlet coupled to a distal end of the cannula, and an atraumatic tip attachable to the blood pump. At least a portion of the atraumatic tip may be configured to be resorbable and/or dissolvable.

In some embodiments, a method may be provided. The method may include inserting a blood pump system to a desired position within a patient. The blood pump system may include a blood pump and an atraumatic tip attachable to the blood pump. The atraumatic tip may have a proximal portion and a distal portion. The method may include resorbing and/or dissolving at least a distal portion of the atraumatic tip into the patient's body.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 shows a view of a blood pump system;

FIG. 2 shows an illustrative side view of a portion of a resorbable atraumatic tip;

FIG. 3 shows an illustrative cross-sectional side view of a portion of a resorbable atraumatic tip;

FIG. 4A shows an illustrative side view of a portion of a resorbable atraumatic tip;

FIG. 4B shows an illustrative side view of a portion of a resorbable atraumatic tip;

FIG. 4C shows an illustrative side view of a portion of a resorbable atraumatic tip;

FIG. 4D shows an illustrative side view of a portion of a resorbable atraumatic tip; and

FIG. 5 depicts a method according to one embodiment.

### DETAILED DESCRIPTION

As it is known, percutaneous blood pumps may be inserted into a patient's vasculature to assist with cardiovascular function. As it is also known, such blood pumps may include atraumatic tips to assist with maintaining inlet position in the patient's ventricle by providing a touch point against ventricular structures and damping motion of the pump during the cardiac cycle. As it is also known, atraumatic tips also may mitigate the chances of unintentional dislodgement of the inlet from the ventricle during positioning of the pump in the patient.

However, in some instances, such atraumatic tips may provide ample stagnant surface area in the patient's ventricle and it is possible that thrombogenesis during periods of long-term support. For purposes herein, thrombogenesis includes the clotting of blood. In such instances, when thrombogenesis occurs, clots may be released into the patient's blood and such clots may enter the blood pump. In other instances, thrombogenesis may occur around the atraumatic tip and may cause the atraumatic tip to become attached to anatomy in the patient's heart.

Accordingly, the inventors have recognized a need to provide the benefits of an atraumatic tip during delivery and positioning while alleviating the problems associated with such atraumatic tips during long-term support.

According to one aspect of the present disclosure, a blood pump with an atraumatic tip may be provided. The atraumatic tip may comprise a distal portion and a proximal portion. In one embodiment, the atraumatic tip may be made from a material configured to be resorbable. The material may allow the atraumatic tip to be configured to passively reabsorb into the patient's blood while located inside of the patient. In some embodiments, the atraumatic tip may be made from a material configured to be dissolved into the blood of a patient and thus excreted out of the patient.

In some embodiments, the atraumatic tip may include a distal portion and a proximal portion. In one embodiment, the entire atraumatic tip may be configured to be resorbable and/or dissolvable. In other embodiments, either the distal portion or the proximal portion may be configured to be resorbable and/or dissolvable. Alternatively, the distal and proximal portions may be configured to resorb and/or dissolve at different rates over time.

In one embodiment, the atraumatic tip may comprise an exterior wall and an interior wall extending from the distal portion to the proximal portion forming a lumen therethrough. The exterior layer may be configured to be resorbable and/or dissolvable. In some embodiments, the atraumatic tip may comprise one or more interior layers. At least one of the interior layers may be configured to be resorbable and/or dissolvable.

In one embodiment, the atraumatic tip may be disconnected from the blood pump. The atraumatic tip may be configured to passively resorb and/or dissolve.

In a method of resorbing and/or dissolving an atraumatic tip, in particular the atraumatic tip described herein, the blood pump may be inserted into the patient and the position of the atraumatic tip is maintained. During the maintenance of the position, the atraumatic tip may be exposed to blood and resorbs and/or dissolves over a period of time while it is inside of the patient. In another embodiment, the blood pump may be inserted into the patient and the atraumatic tip may be resorbed and/or dissolved after the pump is removed at the portion of support.

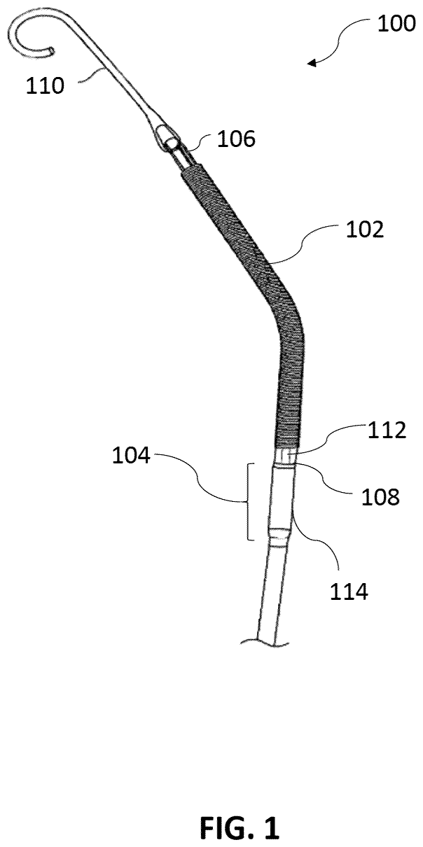

Turning to the figures, FIG. 1 shows a view of a blood pump system 100. The blood pump system 100 may include a cannula 102, a blood pump 104, a blood flow inlet 106, a blood flow outlet 108, and an atraumatic tip 110. The cannula 102 may be connected to the blood flow inlet 106 on the distal portion and the blood flow outlet 108 at the proximal portion of the cannula 102. The blood pump 104 may include an impeller 112 which may be disposed inside of the blood flow outlet 108. In some embodiments, the blood pump 104 may further include an on-board motor 114. In other embodiments, the blood pump 104 may include an extracorporeal motor. The atraumatic tip 110 may assist with positioning the blood pump assembly 100 within the patient and also with stabilizing the blood pump assembly 100 during operation. In some embodiments, the blood pump system 100 may be positioned in the left ventricle of a patient. In other embodiments, the blood pump system 100 may be positioned in the right ventricle of a patient.

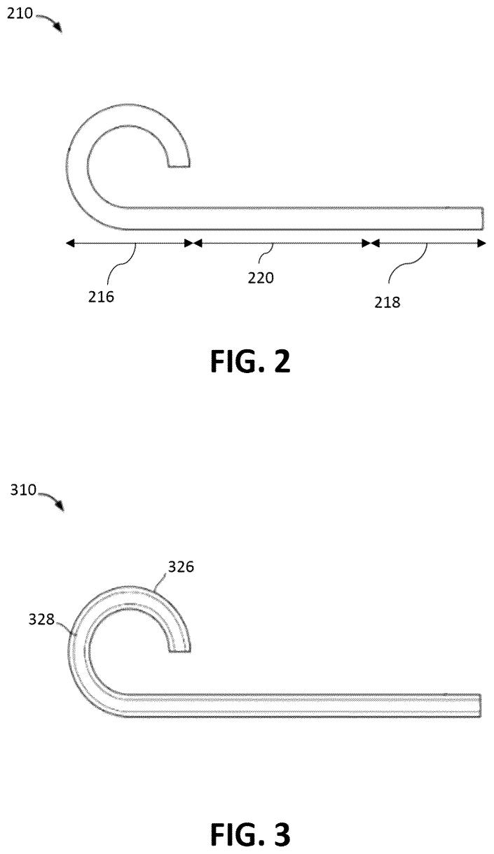

As seen in FIG. 2, the atraumatic tip 210 may include a proximal portion 216 and a distal portion 218. The distal portion 218 may be 25-75% of the total length of the atraumatic tip 110. In other embodiments, there may be a central portion 220 positioned between the distal portion 218 and the proximal portion 216. The distal portion 218, proximal portion 216, and the central portion 220 may be 25%, 50%, and 25% of the total length of the atraumatic tip 210, respectively. As will be appreciated, although described as having the aforementioned proportions, the distal portion 218, proximal portion 216, and the central portion 220 may be of any suitable proportions.

In some embodiments, the distal portion 218 may be configured to be in a pigtail shape. In some embodiments, distal portion 218 may be configured to be in a “J” shape. In other embodiments, the distal portion 218 may be configured in any curved shape. The distal portion 218 may aid in stabilizing the pump within the ventricle and may aid in avoiding suction of heart tissue into the pump during operation.

In one embodiment, the atraumatic tip 110 may be configured to be resorbable. The atraumatic tip 110 may passively resorbs into the patient's blood over time during support. In other embodiments, the atraumatic tip 110 may be configured to dissolve into the patient's blood. The dissolved matter may later be excreted through the patient. In some embodiments, the atraumatic tip 110 may be composed of a bioresorbable polymer. The bioresorbable polymer may include for example polylactide (PLA), poly lactide-co-D, L lactide (PLDL), poly lactide-co-glycolide (PLGA), poly lactide-co-caprolactone (PLCL), poly caprolactone (PCL), poly dioxanone (PDO), or poly lactide-co-trimethylene carbonate (PL-TMC). The aforementioned exemplary bioresorbable polymers may have amorphous or semi-crystalline material structures. In other embodiments, the atraumatic tip 110 may be composed of any suitable bioresorbable material. In some embodiments, the bioresorbable polymer may have a degradation time of at least 10 days. In other embodiments, the bioresorbable polymer may have a degradation time of many months, for example up to one year or longer. In other embodiments, the physician may select the atraumatic tip 110 having certain material properties that correlate to the expected duration of support, thus ensuring that the atraumatic tip 110 will resorb and/or dissolve near the end of support.

In some embodiments, the atraumatic tip 110 may be comprised of a material which will reabsorb and/or dissolve into the patient's blood upon introduction of an additional solution into the area surrounding the blood pump assembly 100 and the atraumatic tip 110. In such embodiments, the solution may be configured to break down the atraumatic tip 110 and cause it to be reabsorbed and/or dissolved into the patient's blood.

In some embodiments, only a portion of the atraumatic tip 110 may be configured to be resorbable. In such embodiments, for example, either the distal portion 218 and/or the proximal portion 216 may be configured to be resorbable and/or dissolvable. In certain embodiments, the distal portion 218 may resorb and/or dissolve into the patient's blood during support and the proximal portion 216 does not, thus still providing an atraumatic tip 110 on the distal portion of the blood pump assembly 100 but allowing the surface area of the atraumatic tip 110 to be smaller. In other embodiments, the distal portion 218 and the proximal portion 216 may be composed of different materials such that the rate at which they resorb and/or dissolve is different. For example, in one embodiment, the distal portion 218 may be comprised of one material that resorbs and/or dissolves steadily over time, while the proximal portion 216 may be comprised of a different material which resorbs and/or dissolves quickly closer to the portion of support. Alternatively, the proximal portion 216 may be composed of a material that does not resorb and/or dissolve. In another embodiment, the proximal portion 216 may be composed of a material that becomes flexible in higher temperatures, such as the temperature in the ventricle. For example, the proximal portion 216 may curl into a J-shape or a complete circle after the distal portion 218 has resorbed and/or dissolved, thus providing an atraumatic tip for the remainder of support.

In some embodiments, the atraumatic tip 110 may be removable from the blood pump assembly 100 at the conclusion of support. When the blood pump assembly 100 is positioned within the patient, thrombogenesis may occur. In such case, the atraumatic tip 110 may be removed from the blood pump assembly 100 before removal of the blood pump assembly 100 so as to prevent damage to the patient's anatomy or disrupt the patient's stability. In such embodiments, the atraumatic tip 110 may be composed of any suitable material that is configured to be resorbed and/or dissolved and which is safe to be incorporated into the patient's tissue. In other embodiments, the atraumatic tip 110 may be composed of a material which passively reabsorbs and/or dissolves into the patient's blood.

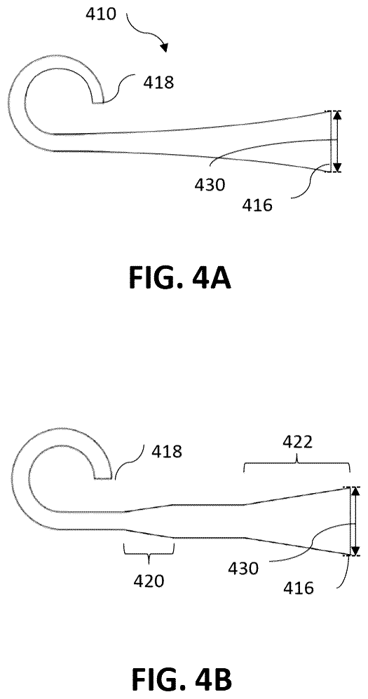

In some embodiments, the atraumatic tip 310 may include an exterior wall an exterior wall 326 and an interior wall 328, as shown in FIG. 3. In such embodiments, the exterior wall 326 and the interior wall 328 may be comprised of different materials. For example, in such embodiments, the exterior wall 326 may be comprised of a resorbable material and the interior wall 328 may be comprised of a material that is not configured to be resorbable. Thus, if thrombogenesis has occurred, the exterior wall 326 may passively reabsorb into the patient's blood and leave the interior wall 328 of the atraumatic tip 310 free from overgrowth. In some embodiments, the atraumatic tip 310 may include more than one interior wall. In such embodiment, each interior wall may be comprised of different materials. For example, the exterior wall 326 may be comprised of a material that resorbs/dissolves quickly, while the interior walls are comprised of materials that resorb/dissolve slowly.

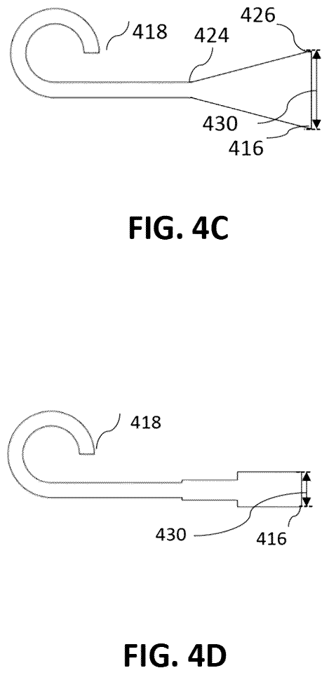

FIGS. 4A-D show illustrative examples of an atraumatic tip 410. In some embodiments, the atraumatic tip may have an outer diameter 430 that changes from the proximal portion 416 to the distal portion 418. In such embodiments, the proximal portion 416 may have an outer diameter 430. In one embodiment, as shown in FIG. 4A, the outer diameter 430 may decrease continuously from the proximal portion 416 in a direction towards the distal portion 418. In other embodiments, the outer diameter 430 may increase continuously from the proximal portion 416 in a direction towards the distal portion 418. In other embodiments, as shown in FIG. 4B, the outer diameter may decrease from the proximal section 416 to the distal section 418 in one or more segments. For example, as show in FIG. 4B, the outer diameter may decrease continuously through a first segment 420, followed by a constant outer diameter for at least a portion of the atraumatic tip 410, and a second segment 422 where the outer diameter may decrease further. As will be appreciated, although two segments are described, any suitable number of segments may be used to decrease the diameter along the length of the atraumatic tip. In some embodiments, the length of the first segment 420 and the second segment 422 may be the same. In other embodiments, the length of the first segment 420 and the second segment 422 may be different. In other embodiments, as shown in FIG. 4C, the outer diameter 430 may decrease in a direction towards the distal portion 418 from a first point 424 to a second point 426 located distal on the atraumatic tip to the first point. As will be appreciated, although the second point 426 is shown at the distal end 416 of the atraumatic tip, the second point 426 may be located at any suitable point distal to the first point 424. In another embodiment, as shown in FIG. 4D, the outer diameter 430 may decrease in a step-wise change from the proximal portion 416 towards the distal portion 418. The outer diameter 430 may decrease continuously, in step-wise changes, or any other suitable design. As will be appreciated, although certain geometries are described herein, the outer diameter may decrease along the length of the atraumatic tip via any suitable geometry. As will further be appreciated, the outer diameter may decrease via any suitable combination of any suitable geometries. As will also be appreciated, although the outer diameter is described as decreasing along the length of the atraumatic tip herein, the outer diameter may increase along the length of the atraumatic tip. While outer diameter is used, there is no requirement that the atraumatic tip have a circular cross-sectional measurement. For example, in some embodiments, the atraumatic tip may be an oval shape and thus the diameter may refer to the largest cross-sectional measurement. As will be appreciated, although the atraumatic tip is shown having a circular cross-sectional geometry, the atraumatic tip may be of any suitable cross-sectional geometry.

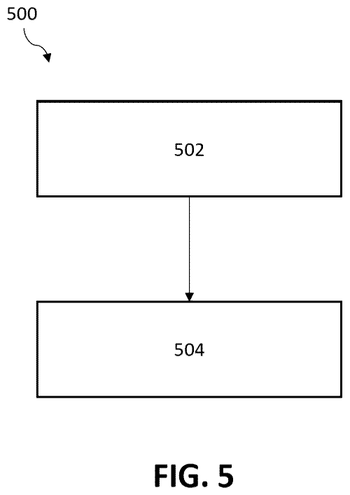

FIG. 5 illustrates a method 500 of inserting 502 a blood pump according to some aspects of the present disclosure. In some embodiments, the method may include the first step of inserting 502 the blood pump assembly 100 into the patient. The blood pump assembly 100 may include an atraumatic tip 110. The method also may include the step of maintaining 504 the position of the blood pump assembly 100 in the patient for a period of time. According to some embodiments, the method may include the step of resorbing and/or dissolving the at least a portion of the atraumatic tip 110 into the patient's blood. The method may also include the step of resorbing and/or dissolving the distal tip 218. Alternatively, the method may also include the distal portion 218 and the proximal portion 216 being configured to resorb and/or dissolve at different rates over time. In other embodiments, the method may also include disconnecting the atraumatic tip 110 from the blood pump assembly 100. The method may also include a step where the blood pump assembly 100 is removed from the patient after the atraumatic tip 110 is disconnected.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Various embodiments may be described below.

In a first embodiment, an atraumatic tip for a blood pump system may be provided. The atraumatic tip may include: a proximal portion attachable to a blood pump; and a distal portion, wherein at least the distal portion of the atraumatic tip is configured to be resorbable and/or dissolvable into a body of a patient.

In a second embodiment, based on the first embodiment, each of the distal and proximal portion are resorbable or dissolvable.

In a third embodiment, based on the first or second embodiments, the entire atraumatic tip is resorbable or dissolvable.

In a fourth embodiment, based on any of the first through third embodiments, the distal and proximal portion are configured to be passively resorbed over time.

In a fifth embodiment, based on any of the first through fourth embodiments, the distal and proximal portion being configured to be passively reabsorbed at different rates over time.

In a sixth embodiment, based on any of the first through fifth embodiments, the atraumatic tip is configured to be actively dissolved for resorption in a body of a patient and/or excretion from the body of a patient.

In a seventh embodiment, based on any of the first through sixth embodiments, the atraumatic tip further includes an exterior layer, the exterior layer extending at least the longitudinal length of the distal portion, an interior layer arranged within the exterior layer and corresponding to at least a portion of the longitudinal length of the exterior layer.

In an eighth embodiment, based on the seventh embodiment, at least the exterior layer is configured to be resorbable or dissolvable.

In a ninth embodiment, based on the seventh or eighth embodiment, the atraumatic tip comprises one or more interior layers.

In a tenth embodiment, based on the ninth embodiment, at least one of the interior layers is resorbable or dissolvable.

In an eleventh embodiment, based on any one of the first through tenth embodiments, the distal portion and the proximal portion each comprise at least one of polylactide, poly lactide-co-D, L lactide, poly lactide-co-glycolide, poly lactide-co-caprolactone, poly caprolactone, poly dioxanone, or poly lactide-co-trimethylene carbonate.

In a twelfth embodiment, based on the eleventh embodiment, the distal portion and the proximal portion are comprised of the same material.

In a thirteenth embodiment, based on the eleventh embodiment, the distal portion and proximal portion are comprised of different materials.

In a fourteenth embodiment, based on any one of the first through the thirteenth embodiments, the atraumatic tip having a central portion extending between the distal portion and the proximal portion.

In a fifteenth embodiment, based on any one of the first through the fourteenth embodiments, the distal portion is j-shaped or pigtail shaped.

In a sixteenth embodiment, based on any one of the first through the fifteenth embodiments, the proximal portion has an outer diameter, wherein the atraumatic tip has an outer diameter that decreases from the proximal portion to the distal portion.

In a seventeenth embodiment, based on any one of the first through the sixteenth embodiments, the outer diameter decreases from the proximal portion in a direction to the distal end.

In an eighteenth embodiment, based on the seventeenth embodiment, the outer diameter decreases continuously from the proximal portion in a direction to the distal portion.

In a nineteenth embodiment, based on the seventeenth or eighteenth embodiment, the outer diameter decreases from the proximal portion to a first point located on the atraumatic tip between the proximal and distal portions.

In a twentieth embodiment, based on the seventeenth embodiment, the outer diameter decreases in a step-wise change from the proximal portion in a direction to the distal end.

In a twenty-first embodiment, based on the nineteenth or twentieth embodiment, the outer diameter decreases from a second point located distal to the first point on the atraumatic tip in a direction to the distal portion.

In a twenty-second embodiment, based on any of the first through twenty-first embodiments, the distal portion of the atraumatic tip is between 25% and 75% of a total length of the atraumatic tip.

In a twenty-third embodiment, based on the twenty-second embodiment, the distal portion of the atraumatic tip is 25% of a total length, the central portion is 50% of the total length, and the proximal portion is 25% of the total length.

In a twenty-fourth embodiment, based on any of the first through twenty-third embodiments, the atraumatic tip is removably connected to the blood pump.

In a twenty-fifth embodiment, a blood pump system may be provided, that may include: (i) a blood pump, the blood pump comprising: (a) a cannula; and (b) a blood flow inlet coupled to a distal end of the cannula; and (ii) an atraumatic tip attachable to the blood pump; wherein at least a portion of the atraumatic tip is configured to be resorbable and/or dissolvable.

In a twenty-sixth embodiment, based on the twenty-fifth embodiment, the atraumatic tip may include a proximal portion attachable to the blood flow inlet, the atraumatic tip further including a distal portion, wherein at least the distal portion is configured to be resorbable and/or dissolvable.

In a twenty-seventh embodiment, a method may be provided. The method may include: (i) inserting a blood pump system to a desired position within a patient, the blood pump system including a blood pump and an atraumatic tip attachable to the blood pump, the atraumatic tip having a proximal portion and a distal portion; and (ii) resorbing and/or dissolving at least a distal portion of the atraumatic tip into the patient's body.

In a twenty-eighth embodiment, based on the twenty-seventh embodiment, the method may further include the step of maintaining the desired position of the blood pump system, wherein the step of maintaining occurs after the step of positioning.

In a twenty-ninth embodiment, based on the twenty-seventh or twenty-eighth embodiment, the method may further include the step of resorbing and/or dissolving at least a distal portion of the atraumatic tip into the patient's body of a period of time.

In a thirtieth embodiment, based on any of the twenty-seventh through twenty-ninth embodiments, the proximal portion and the distal portion are configured to passively resorb at different rates over time.

In a thirty-first embodiment, based on any of the twenty-seventh through thirtieth embodiments, the method may further include disconnecting at least the distal portion from the blood pump, wherein the step of disconnecting occurs before the step of resorbing and/or dissolving.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An atraumatic tip for a blood pump system, the atraumatic tip comprising:
a proximal portion attachable to a blood pump; and
a distal portion;
wherein at least the distal portion of the atraumatic tip is configured to be resorbable and/or dissolvable into a body of a patient.

2. The atraumatic tip of claim 1, wherein each of the distal and proximal portion are resorbable or dissolvable.

3. The atraumatic tip of claim 1, wherein the entire atraumatic tip is resorbable or dissolvable.

4. The atraumatic tip of claim 1, wherein the distal and proximal portion are configured to be passively resorbed over time.

5. The atraumatic tip of claim 1, wherein the distal and proximal portion being configured to be passively reabsorbed at different rates over time.

6. The atraumatic tip of claim 1, wherein the atraumatic tip is configured to be actively dissolved for resorption in a body of a patient and/or excretion from the body of a patient.

7. The atraumatic tip of claim 1, further comprising an exterior layer, the exterior layer extending at least the longitudinal length of the distal portion, an interior layer arranged within the exterior layer and corresponding to at least a portion of the longitudinal length of the exterior layer.

8. The atraumatic tip of claim 7, wherein at least the exterior layer is configured to be resorbable or dissolvable.

9. The atraumatic tip of claim 7, wherein the atraumatic tip comprises one or more interior layers.

10. The atraumatic tip of claim 1, wherein the distal portion and the proximal portion each comprise at least one of polylactide, poly lactide-co-D, L lactide, poly lactide-co-glycolide, poly lactide-co-caprolactone, poly caprolactone, poly dioxanone, or poly lactide-co-trimethylene carbonate.

11. The atraumatic tip of claim 10, wherein the distal portion and the proximal portion are comprised of the same material.

12. The atraumatic tip of claim 10, wherein the distal portion and proximal portion are comprised of different materials.

13. The atraumatic tip of claim 1, the atraumatic tip having a central portion extending between the distal portion and the proximal portion.

14. The atraumatic tip of claim 1, wherein the distal portion is j-shaped or pigtail shaped.

15. The atraumatic tip of claim 1, wherein the proximal portion having an outer diameter, wherein the atraumatic tip has an outer diameter that decreases from the proximal portion to the distal portion.

16. The atraumatic tip of claim 1, wherein the outer diameter decreases from the proximal portion in a direction to the distal end.

17. The atraumatic tip of claim 1, wherein the distal portion of the atraumatic tip is between 25% and 75% of a total length of the atraumatic tip.

18. The atraumatic tip of claim 1, wherein the atraumatic tip is removably connected to the blood pump.

19. A blood pump system comprising:
a blood pump, the blood pump comprising:
a cannula; and
a blood flow inlet coupled to a distal end of the cannula;
an atraumatic tip attachable to the blood pump;
wherein at least a portion of the atraumatic tip is configured to be resorbable and/or dissolvable.

20. A method comprising:
inserting a blood pump system to a desired position within a patient, the blood pump system including a blood pump and an atraumatic tip attachable to the blood pump, the atraumatic tip having a proximal portion and a distal portion; and
resorbing and/or dissolving at least a distal portion of the atraumatic tip into the patient's body.
